(19) United States
(12) Patent Application Publication
(10) Pub. No.: US 2007/0239238 A1
(43) Pub. Date: Oct. 11, 2007

(54) CONFIGURABLE FINGER COMPRESS

(76) Inventor: Patricia Nausid, Ketchikan, AK (US)

Correspondence Address: BLACK LOWE & GRAHAM, PLLC, 701 FIFTH AVENUE, SUITE 4800, SEATTLE, WA, 98104, US

(21) Appl. No.: 11/733,439

(22) Filed: Apr. 10, 2007

Related U.S. Application Data
(60) Provisional application No. 60/790,632, filed on Apr. 10, 2006.

(51) Int. Cl. A61F 7/00; A61F 7/12; A61F 5/00

(52) U.S. Cl. 607/96; 602/5; 602/22

(57) ABSTRACT

The preferred invention comprises a tube sized and configured to receive a finger for therapeutic treatment. In one form, the invention includes an exterior shell formed from plastic, silicone, or other flexible materials that defines an interior volume for holding a gel or liquid. The gel or liquid may be either heated or chilled in order to stay hot or cold for a period of time that will facilitate treatment.

### PRIORITY CLAIM

This application claims the benefit of U.S. Provisional application Ser. No. 60/790,632, filed Apr. 10, 2006.

### FIELD OF THE INVENTION

This invention relates generally to splints and ice packs used to treat injuries, particularly to fingers and toes.

### BACKGROUND OF THE INVENTION

Fingers and toes are easily injured from impact with another object, such as balls, walls, or mats. The injury often results in swelling and discoloration, especially around the joint areas. Pain and discomfort increases as the swelling increases. Common treatment for such injury includes the application of cold therapy through local application of cold compresses or ice packs. Splinting of the injured finger or toe is also common.

Usual therapy for degenerative conditions such as arthritis includes the application of heat. Heat therapy is normally achieved through warm water immersion, or topical application of a heated compress.

In each case, whether cold or hot, the current applications are less than ideal. Ice packs, hot packs, and similar compresses are generally bulky and do not readily conform to the size and shape of a finger. Treatment often requires the use of both hands to hold the compress against the affected area, and further does not fully contact the affected area, thereby leaving at least a portion untreated. There is therefore a need for an improved method for treating injured fingers and toes with a hot or cold compress.

### SUMMARY OF THE INVENTION

The invention comprises a tube sized and configured to receive a finger for therapeutic treatment. In one form, the invention includes an exterior shell formed from plastic, silicone, or other flexible materials that defines an interior volume for holding a gel or liquid. The gel or liquid may be either heated or chilled in order to stay hot or cold for a period of time that will facilitate treatment.

In some examples of the invention, the device may be formed in varying lengths or diameters, or may be configured to enable it to be custom fit by adjusting a length or diameter.

### BRIEF DESCRIPTION OF THE DRAWINGS

Preferred and alternative embodiments of the present invention are described in detail below with reference to the following drawings:

FIG. 1A is a perspective view of a preferred finger compress;

FIG. 1B is a perspective view of an alternate preferred finger compress;

FIG. 2 is an illustrative view of a preferred finger compress, shown on the hand of a user;

FIG. 3 is an illustrative view of a plurality of preferred compresses shown on a single finger;

FIG. 4 is a top plan view of a sheet for forming a preferred compress in accordance with one example of the invention;

FIG. 5 is a perspective view of a preferred finger compress, shown in an exemplary process of forming a sheet into a tubular compress; and

FIG. 6 is an illustration of an alternative finger compress, shown on a toe.

### DETAILED DESCRIPTION OF THE PREFERRED EMBODIMENT

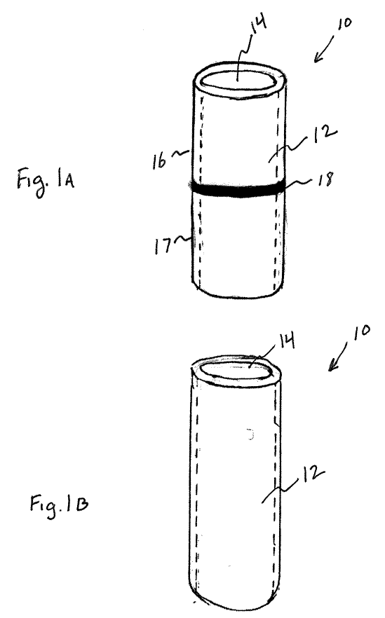

FIG. 1 depicts a preferred form of the finger compress. Within this specification, the invention will generally be referred to as a finger compress. It should be understood that the invention may also be configured for use with toes, and that the use of the term “finger” is not intended to limit the use of the invention to fingers only.

As shown, the compress is substantially tubular in shape, with an internal diameter defined across the span of the internal hollow area. In most examples of the invention, the diameter is smaller than an axial length of the compress, with the axial length extending through middle of the tube and being perpendicular to the diameter. It preferably includes an exterior layer of a flexible material such as a thin sheet of plastic. Any other flexible or elastomeric materials may also be used, and in general the choice of materials is such that it is flexible or semi-flexible at room temperature and is also able to form a leak-proof container for an internal gel or liquid. An internal and an external layer of flexible material is provided, forming an internal volume between the two layers of material.

Although not visible in FIG. 1, the internal volume within the compress is filled with a gel or liquid. Any liquid or gel is suitable within the scope of this invention, and preferably the filler material is able to retain a hot or cold temperature after refrigeration or heating. More preferably, the filler material is able to sustain a cold temperature at which the gel begins to stiffen or freeze into a more solid state so that the compress can serve as a splint as well as a compress. Such gels are currently well known (and further suitable gels may be later developed), and may include mixtures of distilled water, propylene glycol, and thickening agents. Other components such as antibacterial agents may also be included.

The compress 10 includes an external surface 12 and an internal surface 14 defining a volume between the two surfaces. The two surfaces 12, 14 form the internal and external surfaces of a cylinder with a filler material retained within the volume defined by the surfaces. The filler material is preferably a gel or a liquid that is able to retain thermal energy when heated and is also able to substantially maintain a cold temperature when subjected to such a cold temperature for a sufficient period to enable the compress to serve as a cold compress. In alternate forms of the invention, different filler materials are used in hot and cold compress versions in order to provide a first version of a compress that is especially well adapted for use as a cold compress and an second version of a compress that is more particularly adapted for use as a heat compress.

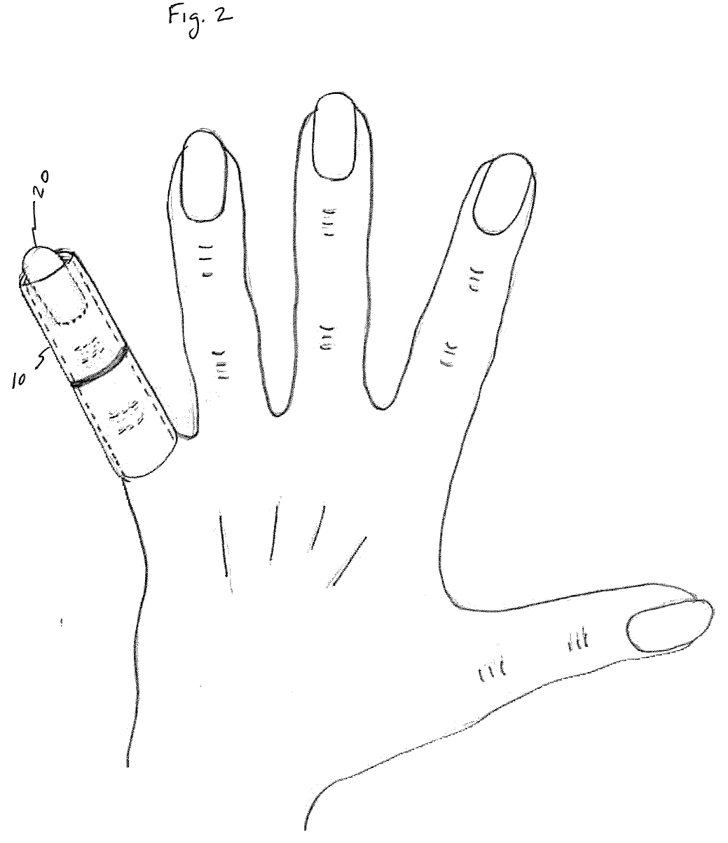

FIG. 2 shows an exemplary use of a preferred compress. As shown a compress 10 (in the form as shown in FIG. 1A) surrounds a first finger 20. Additional compresses may be used on any of the other fingers, and such additional compresses may be the same size, or may be tailored sizes that are configured to accommodate fingers of different sizes. If the gel is properly formulated and refrigerated to reach a solid or nearly solid state, the compresses may each serve as a splint while acting as a compress.

In other examples of the invention, the skin or material of the compress forming the external surface 12 is formed from a semi-rigid or rigid material so that the compress can act as a splint at any temperature, whether hot or cold. In this example, a thicker or more rigid plastic is preferred.

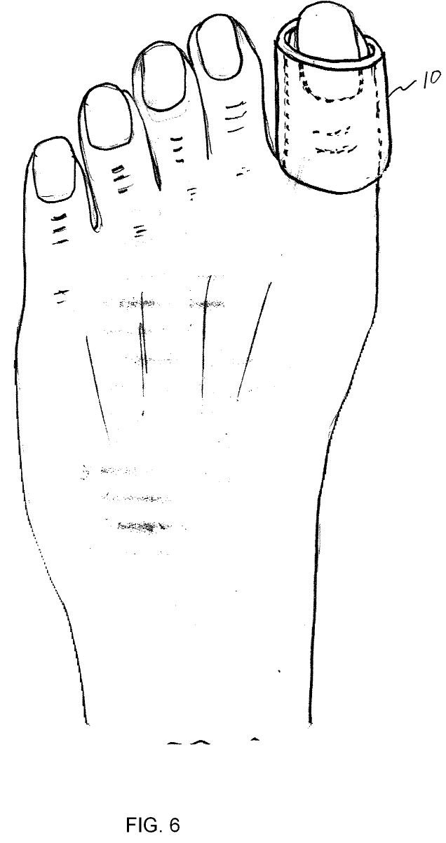

Although FIG. 2 depicts the compress in use with fingers on a human hand, the compress of the present invention can be used equally well with toes on a foot, as illustrated in FIG. 6. In some cases, a single compress will be sized and configured so that it can accommodate either fingers or toes. In other cases, the compress is sized and shaped to be tailored to either a finger or a toe.

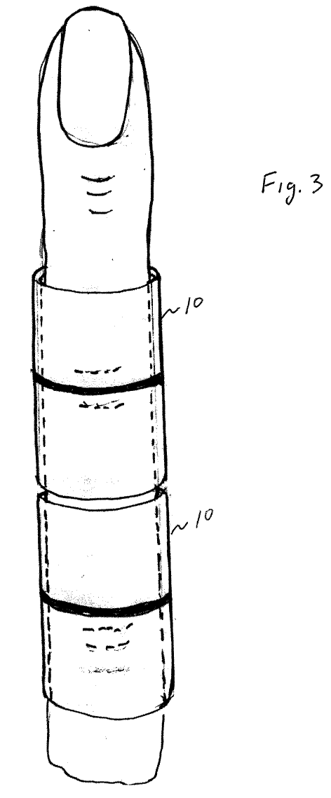

FIG. 3 shows a finger with which two compresses are used. In different forms of the invention, a single compress may be configured to be the length of a particular finger, or may be much shorter to accommodate shorter fingers. The shorter versions as shown are preferably sized to be about the length of one of the phalanges on a typical human finger, or about one inch. Thus, two or three compresses may be used in order to encapsulate an entire thumb or finger with this version of the invention. Longer compresses may be the length of a human finger, including versions that are three inches, four inches, or longer in length.

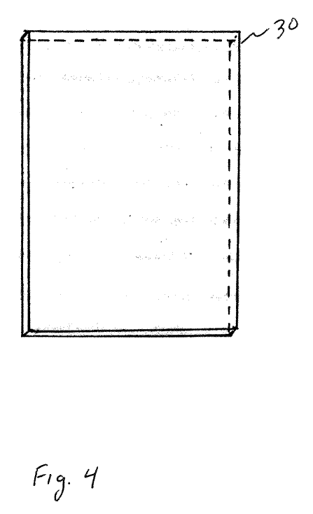

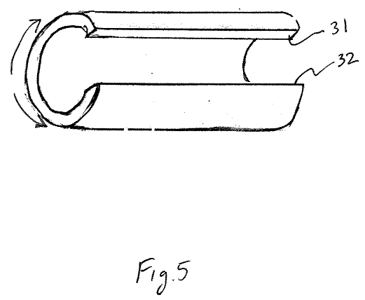

FIG. 4 illustrates a preferred method for producing the compress. Initially a gel-filled sheet 30 is formed, with the sheet being produced in accordance with the description of the preferred compress as described above. In other words, in one example the sheet includes a first layer of a flexible plastic, a second layer of flexible plastic, and an internal filler material comprising a gel. As best seen in FIG. 5, the sheet 30 is rolled in the direction of the arrows to bring the edges 31, 32 adjacent one another. Optionally, the edges may either abut one another or overlap somewhat. The edges are then secured to one another by using an adhesive, heat bonding, or other means. In some forms of the invention, the edges are removably and adjustably secured to one another by using, for example, releasable tape, hook and loop fasteners, or other means.

While FIGS. 4 and 5 depict a method of construction that begins with a flat sheet, the compress may be formed using different processes, including processes that fill a volume between inner and outer layers that are preconfigured into the cylindrical configuration as shown in FIG. 1.

FIG. 1B illustrates another form of the invention. In this example, the compress is formed as a plurality of compress segments. In this case, two segments 16, 17 are shown, although any number of segments may be used. Each of the segments is constructed in accordance with the compress as described above, but the segments are separated from one another by a membrane 18 that seals a volume of gel within each segment individually. Thus, in this fashion one of the segments 16 may be removed from the other segment 17 by trimming along the separation region or membrane 18. In one form, in the separation region the plastic skin of both the inner and outer layers are adhered together. Using scissors or a knife (for example), the segment may be trimmed at the separation region 18 to remove the segment 16.

The inclusion of a separation region enables the compress 10 to be trimmed to a desired length. In one version of the invention, the compress includes several segments that together define the length of a finger. For example, a single multi-segment compress may include 3, 4, 5 or 6 segments. In this form, the user may trim as many or as few segments as desired in order to form a compress of a desired length. In some segmented forms of the invention, the segments are of varying sizes.

In order to use the compress, the compress is trimmed (if necessary, for a segmented version) and either heated or refrigerated to a desired temperature. A finger or toe is inserted into the compress and retained in that position for a desired time in order to receive the therapeutic value of the compress.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A compress, comprising:
a tubular compress body having an external layer and an internal layer defining an interior volume therebetween, the internal layer further defining a hollow central area having a diameter; and
a filler material contained within the interior volume, the filler material being capable of substantially maintaining a cold temperature after refrigeration;
whereby the diameter of the hollow central area is configured to snugly receive a human finger.

2. The compress of claim 1, wherein the filler material comprises a mixture of water, propylene glycol, and a thickening agent.

3. The compress of claim 1, wherein the internal layer and the external layer are each formed from a flexible material.

4. The compress of claim 1, wherein the filler is a liquid at room temperature and freezes to at least a partially solid state at 32 degrees Fahrenheit.

5. The compress of claim 1, wherein the compress comprises a length perpendicular to the diameter, the length being approximately equal to one human phalange.

6. The compress of claim 1, wherein the compress comprises a length perpendicular to the diameter, the length being approximately one inch.

7. The compress of claim 1, wherein the compress comprises a length perpendicular to the diameter, the length being approximately equal to one human finger.

8. The compress of claim 1, wherein the compress comprises a length perpendicular to the diameter, the length being approximately three inches.

9. The compress of claim 1, wherein the compress further comprises a separation region dividing the interior volume into two sub-volumes, whereby the separation region may be cut to produce two separate tubular compresses.

10. A compress, comprising:
a tubular compress body having an external layer and an internal layer defining an interior volume therebetween, the internal layer and external layers each formed from a material that is substantially flexible at room temperature, the internal layer further defining a hollow central area having a diameter; and
a means for substantially maintaining a hot or cold temperature contained within the interior volume;
whereby the diameter of the hollow central area is configured to receive a human finger.

11. A compress, comprising:
a tubular compress body having an axial length and being formed from an external layer and an internal layer defining an interior volume therebetween, the internal layer and external layers each formed from a material that is substantially flexible at room temperature, the internal layer further defining a hollow central area having a diameter; and
a filler material contained within the interior volume, the filler material being capable of substantially maintaining a cold temperature after refrigeration;
whereby the diameter of the hollow central area is smaller than the axial length of the compress.
